(19) United States
(12) Patent Application Publication
(10) Pub. No.: US 2001/0044600 A1
(43) Pub. Date: Nov. 22, 2001

(54) Closed catheter suction system

(76) Inventor: John I. Elkins, St. James, NY (US)

Correspondence Address: Kelly K. Burris, 2000 Equitable Building, 10 South Broadway, St. Louis, MO, 63102, US

(21) Appl. No.: 09/739,133

(22) Filed: Dec. 18, 2000

Related U.S. Application Data
(60) Provisional application No. 60/204,953, filed on May 17, 2000.

(51) Int. Cl. A61M 1/00; A61M 5/178

(52) U.S. Cl. 604/119; 604/183

(57) ABSTRACT

The present invention relates to closed catheter suction systems used to aspirate secretions from the trachea of a patient and to rinse the catheter after such aspiration. The apparatus comprises a flexible suction catheter encased within a sheath that is fixed to a vacuum source at its proximal end and extendable through an adapter assembly at its distal end, near the patient. The adapter assembly connects the closed catheter suction system to a patient connector, and a suction control valve at the proximal end operates to aspirate secretions and to rinse the catheter. The closed catheter suction system also includes a lavage port and a rinse port, the latter of which is positioned near the distal end and is isolated from the ventilation circuit through the innovative use of a rinse chamber and a one-way valve. Advantageously, the lavage port is positioned near the proximal end, away from the patient, so as to prevent the accidental leakage of lavage into the ventilation circuit. Further, both the lavage port and the rinse port include a self-closing fill-valve that prevents the accidental loss of ventilator volume and which also prevents the discharge of infectious pathogens to the surrounding environment throughout the aspiration and rinse procedures.

### CROSS-REFERENCE TO RELATED APPLICATIONS

This application is based upon Provisional Patent Application, Serial No. 60/204,953, entitled “Integrated Respiratory/Ventilation System For Improved Patient Care”, filed May 17, 2000, the contents of which are incorporated herein by reference in their entirety and continued preservation of which is requested.

### BACKGROUND OF THE INVENTION

1. Field of the Invention

The present invention relates to devices and methods used in respiratory ventilation circuits, and more particularly to closed catheter suction systems which provide for aspiration of secretions from the passageway of a patient in addition to cleansing or rinsing of the catheter after the aspiration procedure is completed. More specifically, the present invention relates to closed catheter suction systems which are adapted for use with endotracheal or tracheal tubes for clearing secretions from the trachea of a patient.

2. Prior Art

Known prior art closed catheter suction systems are largely used for insertion into canals, vessels, passageways, or body cavities of a patient to permit injection of a solution and withdrawal of infectious secretions to maintain a clear and open passageway. The closed catheter suction system of the prior art generally includes a vacuum source at its proximal end (operator end) for providing suction, an adapter at its distal end (patient end) for attachment to an endotracheal tube adapter, and a suction catheter positioned between the proximal and distal ends for aspirating secretions. The suction catheter is generally encased within a clear sheath that seals the exterior of the catheter from external contaminants and seals the surrounding environment from secretions aspirated through the suction catheter. In use, the suction catheter aspirates secretions by traversing the same passageway that supplies oxygen to the patient through the trachea and as such interrupts the flow of oxygen during the aspiration procedure. Accordingly, caution must be observed to complete the aspiration procedure in the shortest amount of time in order to reduce the risk of patient hypoxia.

To thin secretions coated along the trachea of a patient, a saline lavage is typically instilled through a lavage port in the closed catheter suction system for delivery along the trachea by the suction catheter. (Lavage generally refers to the therapeutic washing out of an organ.) After the saline lavage loosens the secretions coated along the trachea, the vacuum source is activated such that the secretions and the saline lavage are both aspirated from the trachea and through the suction catheter to the vacuum source for proper disposal. The interior passageway of the suction catheter, otherwise known in the art as a lumen, is then rinsed with a second injection of lavage, or rinse solution, to prevent obstruction of the lumen during subsequent aspiration procedures. Unfortunately, the rinsing procedure that follows patient aspiration increases the risk of hypoxia since the flow of oxygen to the patient remains interrupted.

Several prior art closed catheter suction systems equipped with rinse ports interrupt the supply of oxygen to the patient during the rinse procedure, and as a result, increase the risk of patient hypoxia because the rinse port is not isolated from the ventilation circuit. For example, U.S. Pat. Nos. 5,449,348 and 5,529,756 to Dryden, and U.S. Pat. No. 5,279,549 to Ranford, disclose rinse ports positioned at the proximal end of the closed catheter suction system which are not isolated from the ventilation circuit. The rinse ports of these prior art devices are in constant fluid communication with the catheter lumen, and no provisions are disclosed which isolate the catheter lumen from the ventilation circuit to prevent interruption of oxygen flow to the patient.

U.S. Pat. No. 5,125,893 to Dryden does disclose a suction catheter which can be removed from the ventilation circuit adapter through a one-way valve, however, no device or method for an isolated rinsing procedure is disclosed. If a rinsing procedure were attempted with the Dryden device, the lavage solution and the infectious secretions would likely exit the tip of the catheter and enter the sheath, resulting in possible contamination of the ventilation circuit since Dryden concedes that some leakage through the one-way valve will occur. Another prior art reference, U.S. Pat. No. 5,333,606 to Schneider et al., discloses a fluid entry port positioned at the distal end of the closed catheter suction system which can only be isolated from the ventilation circuit through the use of a special adapter. The special adapter, not the catheter, causes the normally closed one-way valve to open, which necessarily requires an additional ventilation circuit component. The special adapter is undesirable because connecting and disconnecting accessories to and from the ventilation circuit places the sterile environment at a substantial risk of contamination. Moreover, if the fluid entry port were used as a lavage port to clear secretions, the lavage could flow into the ventilator circuit, thereby resulting in undesirable moisture accumulation and potential bacterial growth. Accordingly, an additional disadvantage of a rinse port which is not isolated from the ventilation circuit is that the rinse solution instilled through the port may inadvertently flow into the ventilation tubing and ultimately compromise the health of the patient.

Rinse ports disclosed in the prior art are often disadvantageously positioned at the proximal end of the closed catheter suction system which requires the rinse solution to travel the entire length of the suction catheter to the distal end thereof and then travel back to the vacuum source located at the proximal end. Such a procedure presents the risk of transporting any residual infectious secretions within the lumen of the suction catheter back into the patient when the rinse solution is instilled. If the infectious secretions do in fact enter the patient, the secretions must be suctioned back out of the patient and the lumen must again be rinsed, resulting in a vicious cycle of contamination and an extremely inefficient procedure. Since rinsing procedures generally occur immediately after the aspiration procedure when the patient is hypoxic, several cycles of suctioning and rinsing can compromise the health of the patient over an extended period of time.

For example, U.S. Pat. Nos. 5,449,348 and 5,125,893 issued to Dryden, U.S. Pat. No. 5,139,018 to Brodsky et al., and U.S. Pat. No. 5,083,561 to Russo disclose rinse ports located at the proximal end of the closed catheter suction system for a “down-the-catheter lumen purge.” Additional prior art references that disclose rinse ports at the proximal end of the closed catheter suction system include U.S. Pat. No. 5,279,549 to Ranford, U.S. Pat. No. 5,139,018 to Brodsky et al., U.S. Pat. No. 5,083,561 to Russo, and U.S. Pat. No. 5,073,164 to Hollister et al. Regrettably, the prior art is deficient because the location of the rinse port along the closed catheter suction system presents the risks of transporting residual infectious secretions back into the patient and inducing severe hypoxia in the patient from the necessitous cycle of aspiration and rinsing.

Turning now to the lavage ports of the prior art, many known catheter systems such as those found in U.S. Pat. No. 4,850,350 to Jackson or U.S. Pat. No. 4,834,726 to Lambert disclose lavage ports positioned at the distal end of the closed catheter suction system near the patient connector. Unfortunately, a lavage port located at the distal end of a closed catheter suction system may introduce lavage into the ventilation tubing if the ventilator circuit and the closed catheter suction system are not positioned properly. For example, when the closed catheter suction system is placed in certain positions, gravity often causes the saline lavage to inadvertently enter the ventilator tubing instead of the endotracheal or tracheostomy tube. If the saline lavage enters the ventilation tubing, the resulting moisture along the tubing can promote undesirable bacterial growth and compromise the health of the patient.

Prior art catheter systems such as those assigned to Ballard Medical Products in U.S. Pat. Nos. 4,834,726, 4,696,296, 4,938,741, 4,569,344, and 4,638,539 disclose a lavage port connected to a closed catheter suction system that includes a cap to seal the ventilation circuit after lavage is instilled. When the cap is inadvertently left open or off, partial loss of ventilator volume to the patient can result and infectious pathogens from the patient could also be exuded from the open port. Moreover, partial loss of ventilator volume can lead to hypoxia and result in an increased risk to the health of the patient. If infectious pathogens are exuded from the open port, the health of surrounding medical staff and visitors are also placed at risk.

Some of the prior art closed catheter suction systems also include a locking valve that is designed to prevent accidental suctioning by the patient such as those disclosed in U.S. Pat. No. 4,569,344 to Palmer. Unfortunately, the extra safety step to lock the valve is often overlooked by medical staff, therefore further compromising the health of the patient if the patient were to accidentally activate the valve under the mistaken belief that the locking valve was the nurse call bell.

Generally, the suction catheter of the known art closed catheter suction systems projects through a seal and directly into the ventilator circuit above the endotracheal or tracheostomy tube even when fully retracted. If a patient or member of the medical staff were to pull on the catheter system, the outer envelope or sheath could tear, causing the catheter to be pulled from the ventilation circuit and thereby causing a leak in the circuit and inhibiting the requisite delivery of oxygen to the patient. Excessive time in correcting the leak can result in severe hypoxia, hypercapnia, or cardiac arrest in a patient.

Accordingly, a need exists in the art for a closed catheter suction system which maintains proper ventilation volume to the patient during the rinsing procedure that follows aspiration of secretions from the passageway of the patient. A further need exists for a closed catheter suction system that prevents lavage or rinse solution from entering the ventilation circuit during both aspiration and rinsing procedures in order to reduce the risk of bacterial growth in the ventilation circuit. Yet a further need exists for a closed catheter suction system which prevents accidental suctioning by the patient. Additionally, a need exists for a closed catheter suction system which prevents loss of ventilator volume and the spread of infectious pathogens through the lavage port(s) of the system.

### OBJECTS AND SUGARY OF THE INVENTION

The primary object of the present invention is to provide a closed catheter suction system which prevents loss of ventilator volume during the procedure that rinses the lumen of the suction catheter.

Another object of the present invention is to provide a closed catheter suction system that prevents aspirated secretions from re-entering the passageway of the patient during subsequent rinsing of the suction catheter lumen.

A further object of the present invention is to provide a closed catheter suction system which prevents a lavage solution from inadvertently entering the ventilation circuit and causing bacterial contamination.

Yet a further object of the present invention is to provide a closed catheter suction system that prevents the accidental loss of ventilator volume through the lavage port(s).

Another important object of the present invention is to provide a closed catheter suction system which prevents the discharge of infectious pathogens through the lavage port(s).

Another further object of the present invention is to provide a closed catheter suction system that prevents accidental patient suctioning.

Yet another object of the present invention is to provide a closed catheter suction system which prevents accidental leakage from a ventilation circuit if the catheter system is mishandled by the patient or medical staff.

In brief summary, the present invention overcomes and substantially alleviates the deficiencies in the prior art by providing a closed catheter suction system that isolates the rinse procedure while maintaining the integrity and sterile environment of the ventilation circuit. The closed catheter suction system of the present invention comprises a flexible suction catheter encased within a sheath that is extendable through a patient connector attached at the distal end of the closed catheter suction system and which is in fluid communication with a vacuum source attached at the proximal end thereof. An adapter assembly is provided at the distal end of the closed catheter suction system to establish a connection for fluid communication between the suction catheter and the patient connector. A suction control valve is located at the proximal end of the closed catheter suction system to provide for selective suctioning, or aspiration, of secretions through the suction catheter.

The suction catheter is fixably attached to the proximal end of the closed catheter suction system and is slidably engaged with the distal end. More specifically, the suction catheter defines a proximal end fixably attached to the suction control valve, and a distal end slidably engaged with the adapter assembly. The suction catheter further defines a lumen through which both lavage and rinse solutions may pass. Additionally, the distal end of the suction catheter defines a tip which includes a plurality of radial openings and/or an axial opening to communicate with the lumen.

The sheath which completely encases the suction catheter is fixably attached to each end of the closed catheter suction system, thereby preventing any undesirable discharge of infectious pathogens from the closed catheter suction system to the surrounding area where medical staff are present. Additionally, both the suction catheter and the sheath are made of a transparent material so that a user can monitor the proper flow of lavage solution, secretions, and rinse solution passing through the lumen of the suction catheter.

The adapter assembly which provides the connection between the suction catheter and the patient connector further comprises a one-way valve that permits ingress and egress of the suction catheter through the patient passageway while maintaining the integrity of the ventilation circuit. Further, a rinse chamber is provided to isolate the suction catheter from the ventilation circuit during the rinse procedure. Both the one-way valve and the rinse chamber are adapted for use with a patient connector as shown and described in Provisional Patent Application, Serial No. 60/204,953, entitled “Integrated Respiratory/Ventilation System For Improved Patient Care”, filed May 17, 2000.

The suction control valve that operates to aspirate secretions further includes a housing that defines an internal chamber, wherein a valve block is disposed between a spring and a cover of the suction control valve. An external groove is formed around the periphery of the valve block and is placed in alignment with the catheter lumen and the vacuum source when the valve block is fully depressed against the spring, resulting in a continuous fluid path between the catheter lumen and the vacuum source. When the valve block is released, the spring normally biases the valve block such that the continuous passageway between the catheter lumen and the vacuum source is closed to fluid flow communication.

The closed catheter suction system of the present invention further comprises two ports in fluid communication with the suction catheter, namely, a lavage port and a rinse port. The lavage port is positioned at the proximal end of the closed catheter suction system and accommodates the instillation of a lavage solution through the catheter lumen for delivery to the passageway of the patient. The rinse port is positioned at the distal end of the closed catheter suction system and advantageously isolates the rinsing procedure from the ventilation circuit. Both of the ports include a fill valve that is self-closing, such as the sealing element disclosed in U.S. Pat. No. 5,641,184 to Mortensen. Importantly, the rinse port is in fluid communication with the rinse chamber of the adapter assembly, such that when the tip of the catheter is disposed within the rinse chamber, a rinse solution can be instilled through the rinse port and into the rinse chamber for rinsing the lumen as more fully described below.

In operation, the adapter assembly of the present invention is attached to a patient connector, such as a safety lock connector disclosed in Provisional Patent Application, Serial No. 60/204,953, entitled “Integrated Respiratory/Ventilation System For Improved Patient Care”, filed May 17, 2000, while the suction control valve is attached to the vacuum source. In this fully retracted position, the tip of the suction catheter is positioned within the rinse chamber of the adapter assembly. Initially, a user grips the proximal end of the closed catheter suction system with one hand and stabilizes the patient connector at the distal end thereof with the other hand. The proximal end is then advanced towards the patient which results in the tip of the suction catheter being passed from the rinse chamber through the one-way valve and the patient connector, and into the passageway of the patient. Ultimately, the tip of the suction catheter and its openings are positioned near or adjacent the secretions which are to be aspirated. A lavage solution is then instilled through the self-closing fill valve in the lavage port, and the solution flows through the lumen and into the passageway of the patient. After the lavage solution has sufficiently loosened the secretions, both the lavage and the secretions are removed, or aspirated, through operation of the suction control valve.

To operate the suction control valve, the user first attaches the suction control valve to a vacuum source and activates the vacuum source. The user then depresses the valve block down against the spring which results in the alignment of the external groove with the lumen and the vacuum source. As a result, a continuous fluid pathway from the patient passageway to the vacuum source is established such that the lavage and secretions can be aspirated through the lumen towards the proximal end of the suction catheter and into the vacuum source for proper disposal.

After the secretions are removed from the passageway of the patient, the lumen must be cleansed for subsequent aspiration procedures so that the residual lavage and secretions within the lumen are not instilled back into the patient. To rinse the lumen after an aspiration procedure, the user first draws back the suction catheter from the passageway of the patient and through the one-way valve until the tip of the suction catheter is positioned within the rinse chamber. After the tip of the suction catheter passes through the one-way valve, the valve automatically closes and ventilation is no longer interrupted so that full ventilator volume can be delivered to the patient during the rinse procedure.

When the tip of the suction catheter is disposed within the rinse chamber, a saline rinse solution is instilled through the self-closing fill valve in the rinse port and into the rinse chamber. The suction control valve is then activated in the same manner described above such that the external groove of the valve block is aligned between the lumen and the vacuum source. Accordingly, the saline rinse solution is suctioned through the openings in the tip of the suction catheter through the lumen and to the vacuum source for proper disposal. As a result, the lumen of the suction catheter is cleared of any residual secretions for subsequent aspiration procedures while the patient remains fully ventilated.

By isolating the rinse procedure from the ventilation circuit, loss of ventilation to the patient is prevented during rinsing as well as in the event that the sheath is torn or pulled apart. Further loss of ventilation and the discharge of infectious pathogens is prevented with the self-closing fill valves provided with both the lavage and rinse ports. Moreover, the advantageous configuration of both the lavage and rinse ports prevents moisture from entering the ventilation circuit and instigating bacterial growth along the inner surface of the ventilation tubing. Accordingly, the closed catheter suction system of the present invention achieves the objects of the invention and overcomes the deficiencies in the prior art.

Additional objects, novel features, and advantages of the present invention will become more apparent to those skilled in the art and are exemplified with more particularity in the detailed description that follows.

### BRIEF DESCRIPTION OF THE DRAWINGS

The above mentioned and other features and objects of the invention, and the manner of attaining them, will become more apparent and the invention itself will be better understood by reference to the following description of an embodiment of the invention taken in conjunction with the accompanying drawings, wherein:

FIG. 1 is an orthogonal view of the closed catheter suction system according to the present invention;

FIG. 2 is an exploded orthogonal view of the closed catheter suction system according to the present invention;

FIG. 3 is an enlarged exploded orthogonal view of the adapter assembly according to the present invention;

FIG. 4 is an enlarged side view of the adapter assembly according to the present invention;

FIG. 5 is a cross-sectional view, taken along the plane of FIG. 4, of the catheter tip positioned within the rinse chamber of the adapter assembly according to the present invention;

FIG. 6 is a cross-sectional view, taken along the plane of FIG. 4, of the catheter tip positioned within the ventilation circuit according to the present invention;

FIG. 7 is an enlarged side view of the suction control valve according to the present invention;

FIG. 8 is an enlarged exploded orthogonal view of the suction control valve according to the present invention;

FIG. 9 is a cross-sectional view, taken along the plane of FIG. 7, of the suction control valve in its open position according to the present invention;

FIG. 10 is a cross-sectional view, taken along the plane of FIG. 7, of the suction control valve in its normally closed position according to the present invention;

FIG. 11 is an enlarged view of the suction control valve cover attached to the suction control valve housing according to the present invention;

FIG. 11A is a cross-sectional view, taken along the plane of FIG. 11, of the housing shoulder engaged with the cover tab according to the present invention;

FIG. 11B is an exploded side view of FIG. 11A, showing the shoulder tab alignment with the tab slot according to the present invention;

FIG. 12 is an enlarged view of the suction control valve spring seated in the valve block cavity according to the present invention;

FIG. 13 is a cross-sectional view, taken along the plane of FIG. 7, of the lavage fitting according to the present invention;

FIG. 14 is a cross-sectional view, taken along the plane of FIG. 4, of the rinse chamber according to the present invention;

FIG. 15 is a side view of the closed catheter suction system initially positioned for aspiration of a patient according to the present invention;

FIG. 16 is a side view of the closed catheter suction system with the catheter extended through the adapter assembly and positioned within the trachea of a patient for aspiration according to the present invention;

FIG. 17 is a side view of the closed catheter suction system with the suction control valve activated and aspirating the passageway of the patient according to the present invention;

FIG. 18 is a side view of the closed catheter suction system with the tip of the catheter positioned within the rinse chamber for rinsing of the lumen according to the present invention; and

FIG. 19 is a side view of the closed catheter suction system with the suction control valve activated and rinsing the lumen according to the present invention.

Corresponding reference characters indicate corresponding parts throughout the several views. Although the drawings represent embodiments of the present invention, the drawings are not necessarily to scale and certain features may be exaggerated in order to better illustrate and explain the present invention.

### DETAILED DESCRIPTION OF THE PRESENT INVENTION

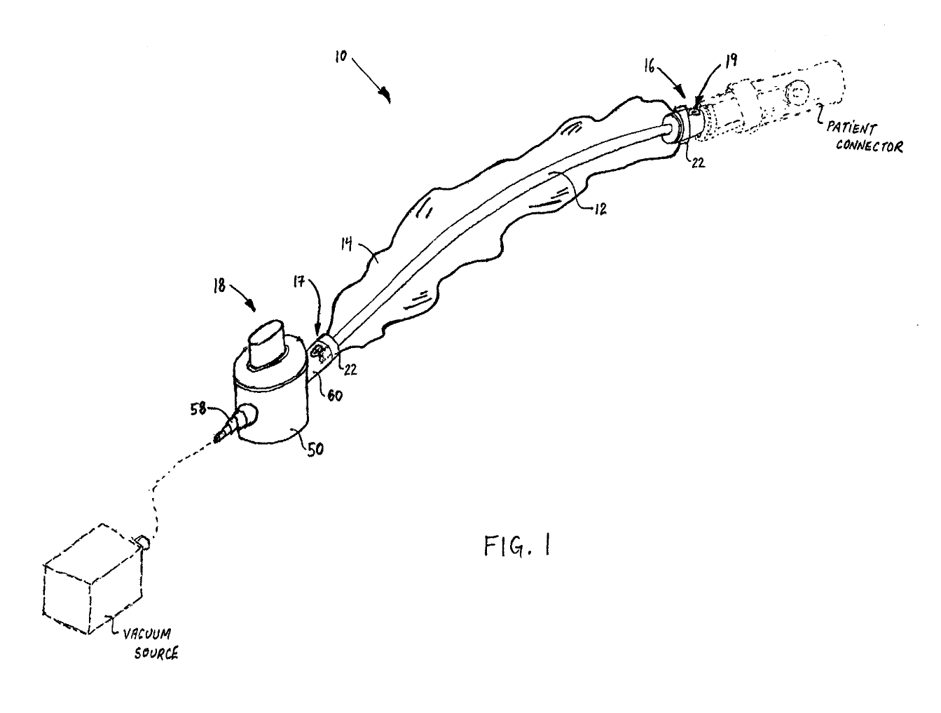

Referring to the drawings, the preferred embodiment of the closed catheter suction system of the present invention is illustrated and generally indicated as 10 in FIG. 1. Closed catheter suction system 10 comprises a flexible suction catheter 12 encased within a sheath 14 that is extendable between an adapter assembly 16 and a suction control valve 18. Adapter assembly 16 is attached to a patient connector (shown in phantom) while suction control valve 18 communicates with a vacuum source (shown in phantom) such that secretions can be aspirated from the patient through catheter 12 to the vacuum source for proper disposal. Closed catheter suction system 10 further includes a lavage port 17 located near the proximal end of catheter 12 for providing a means of introducing a lavage solution through catheter 12 during the aspiration procedure. A rinse port 19 is provided with adapter assembly 16 for the instillation of a rinse solution for cleansing catheter 12 of secretions after completion of the aspiration procedure as shall be described in greater detail below.

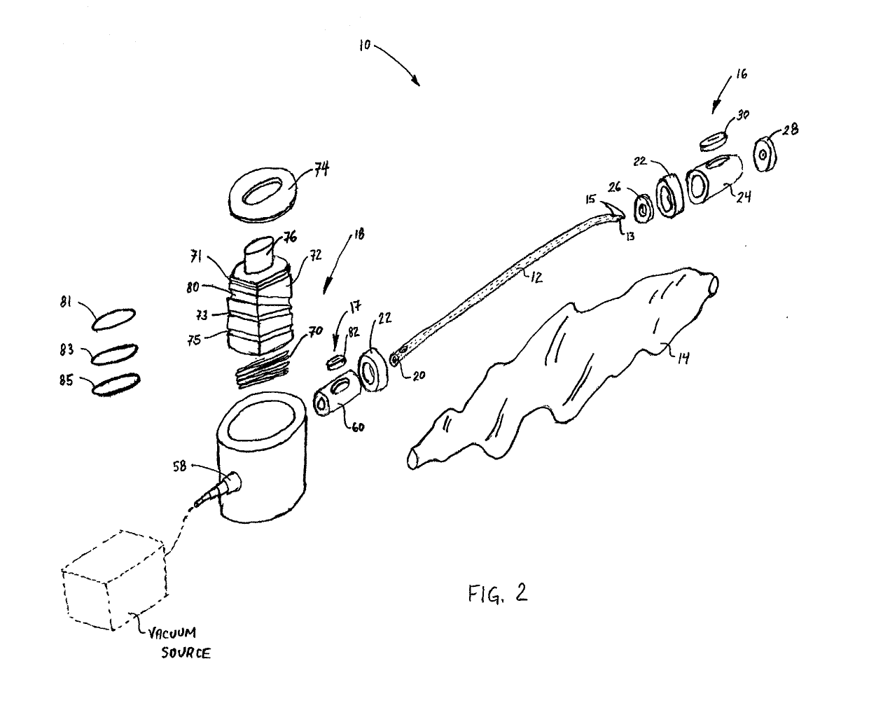

As illustrated in FIG. 2, catheter 12 defines a lumen 20 through which both lavage and rinse solutions pass for aspiration and rinsing procedures, respectively. Catheter 12 further defines a tip 13 having openings 15 to deliver lavage and remove secretions through lumen 20. Sheath 14 completely encases catheter 12 to seal catheter 12 from external contaminants and to prevent leakage of infectious secretions into the immediate area when catheter 12 is withdrawn from the passageway of the patient. Sheath 14 is secured to closed catheter suction system 10 with locking rings 22 positioned adjacent adapter assembly 16 and suction control valve 18. Further, both sheath 14 and catheter 12 are made of a transparent material so that a user can monitor the proper flow of lavage solution, secretions, and rinse solution through lumen 20.

As shown, each of the major components of closed catheter suction system 10, namely, adapter assembly 16, suction control valve 18, lavage port 17, and rinse port 19 include several component parts, the configuration and function of which are now more fully described below.

### Adapter Assembly

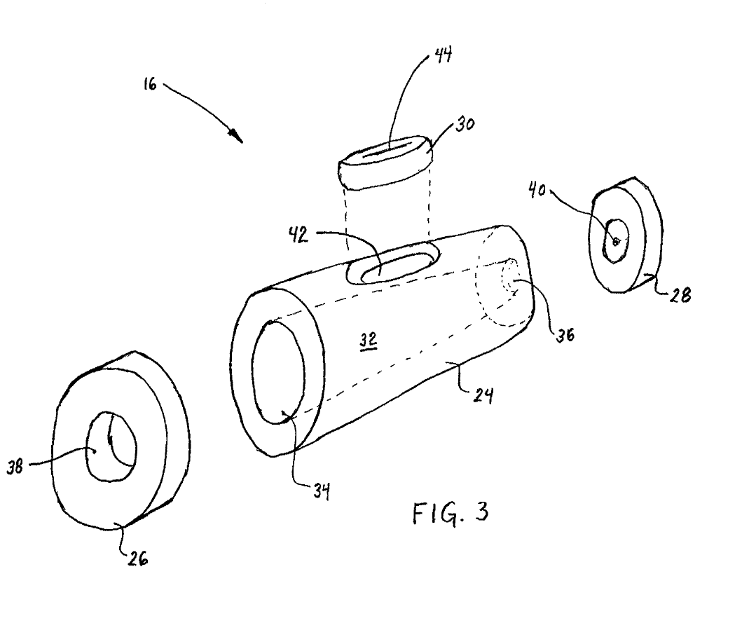

Referring generally to FIG. 2 and to the enlarged detail shown in FIG. 3, adapter assembly 16 comprises a rinse chamber housing 24 engaged with a seal 26, a one-way valve 28, and a fill-valve 30. Rinse chamber housing 24 defines a rinse chamber 32 (shown in partial phantom) with opposed proximal and distal openings 34 and 36, through which catheter 12 is extendable. Seal 26 is positioned adjacent proximal opening 34 and further defines an axial opening 38 which surrounds and seals catheter 12 during operation. One-way valve 28 is positioned adjacent distal opening 36 and further defines a tapered aperture 40 which permits catheter 12 to pass into the ventilation circuit while preventing fluid or volume from passing in the opposite direction. As a result, one-way valve 28 seals the ventilation circuit (not shown) upon withdrawal of catheter 12 from the passageway of the patient, thereby permitting an isolated rinse procedure of catheter 12 as more fully described below.

As shown, rinse chamber housing 24 further defines an upper opening 42 through which fill-valve 30 is seated. Fill-valve 30 is similar to the sealing element disclosed in U.S. Pat. No. 5,641,184 to Mortensen and accordingly includes a slit 44 to accommodate the insertion of syringe or like device for instillation of a rinse solution. Preferably, each of the constituent elements of adapter assembly 16 are adhesively bonded, or joined by other methods known in the art, to rinse chamber housing 24 at each of their respective positions to complete adapter assembly 16.

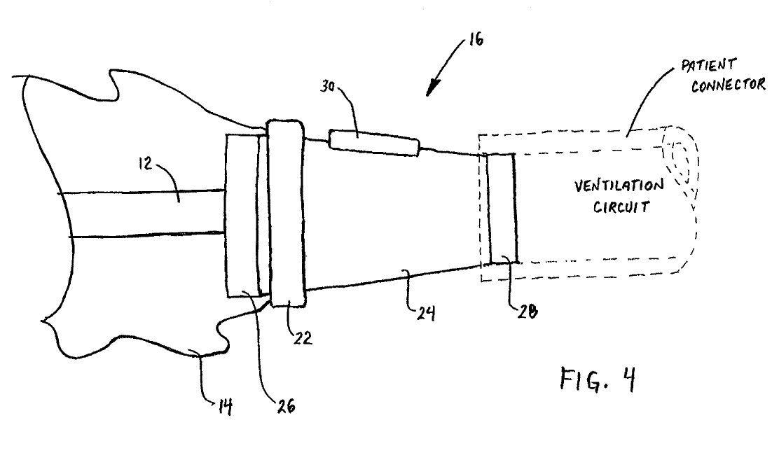

Referring to FIG. 4, adapter assembly 16 is advantageously positioned at the distal end of closed catheter suction system 10 adjacent the patient connector. The distal end of adapter assembly 16 and one-way valve 28 are positioned inside the patient connector, where one-way valve 28 is secured to the interior walls of the patient connector by way of adhesive bonding or other known attachment methods. As shall be appreciated by those of ordinary skill in the art, rinse chamber housing 24 is tapered inward from its proximal end to its distal end to facilitate engagement of adapter assembly 16 to the patient connector. The proximal end of adapter assembly 16 and seal 26 are positioned inside sheath 14 and sheath 14 is secured to the exterior of rinse chamber housing 24 with locking ring 22. Locking ring 22 secures sheath 14 with an interference fit around the periphery of rinse chamber housing 24. The tapered shape of rinse chamber housing 24 also facilitates the attachment of locking ring 22 as ring 22 is slid from the distal end to the proximal end of rinse chamber housing 24 to secure sheath 14 thereto.

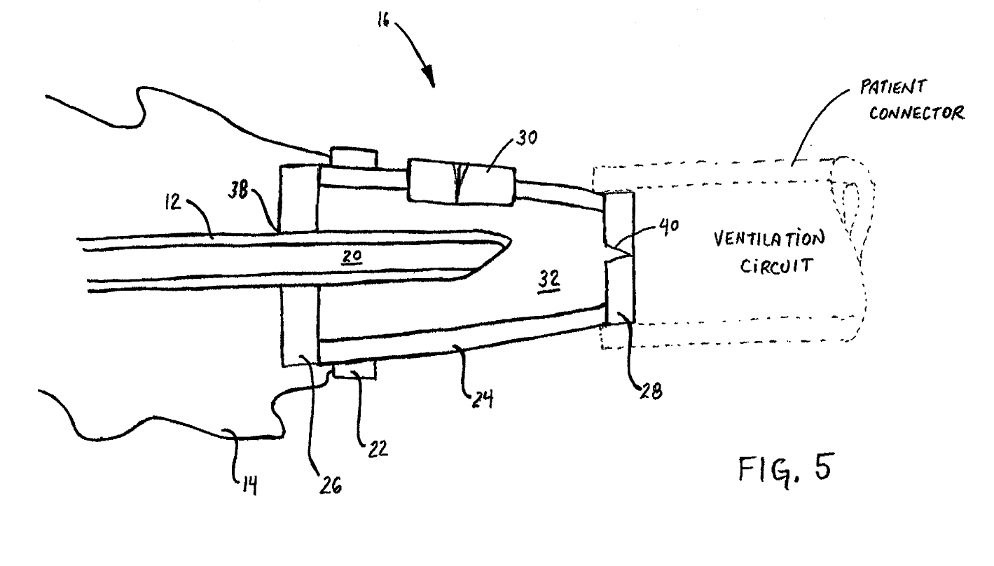

The disposition of catheter 12 within rinse chamber 32 is more clearly illustrated in FIG. 5. When catheter 12 is extended through axial opening 38 and into rinse chamber 32, lumen 20 is exposed to any rinsing solution instilled therein. As further shown, seal 26 prevents fluid or air from exiting rinse chamber housing 24 and entering sheath 14 since axial opening 38 abuts the exterior surface of catheter 12. Similarly, one-way valve 28 seals the ventilation circuit from rinse chamber 32 while permitting catheter 12 to enter the passageway of the patient. Accordingly, the rinse procedure which follows aspiration of the patient is effectively isolated from the ventilation circuit which results in delivery of full ventilator volume to the patient during the rinse procedure.

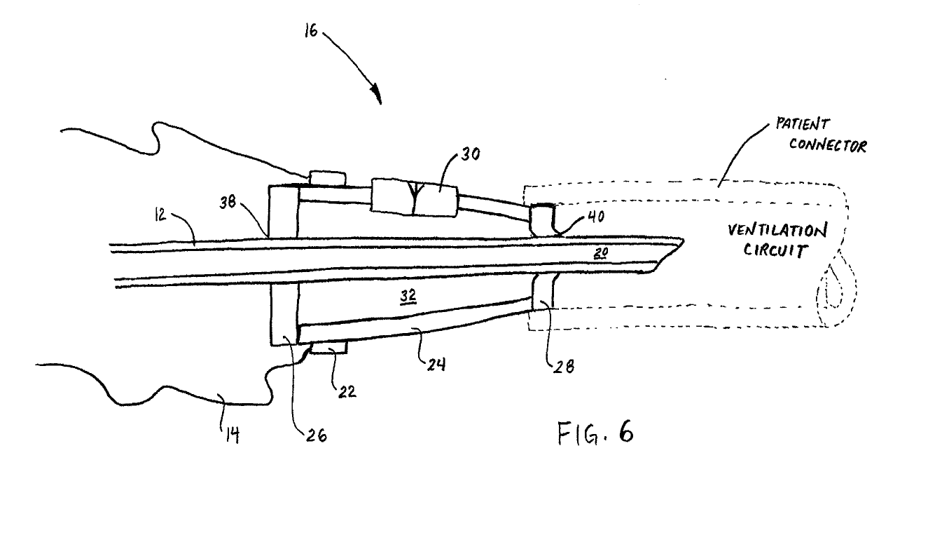

Referring to FIG. 6, when catheter 12 is further advanced through one-way valve 28, tapered aperture 40 opens and permits the passage of catheter 12 into the ventilation circuit while sealing off rinse chamber 32. Because tapered aperture 40 abuts the exterior surface of catheter 12, rinse chamber 32 is sealed from the ventilation circuit while seal 26 prevents any fluid or air from entering sheath 14. As a result, lumen 20 passes into the ventilation circuit and into to the passageway of the patient for subsequent lavage instillation and aspiration as shall be more described in greater detail below.

### Suction Control Valve

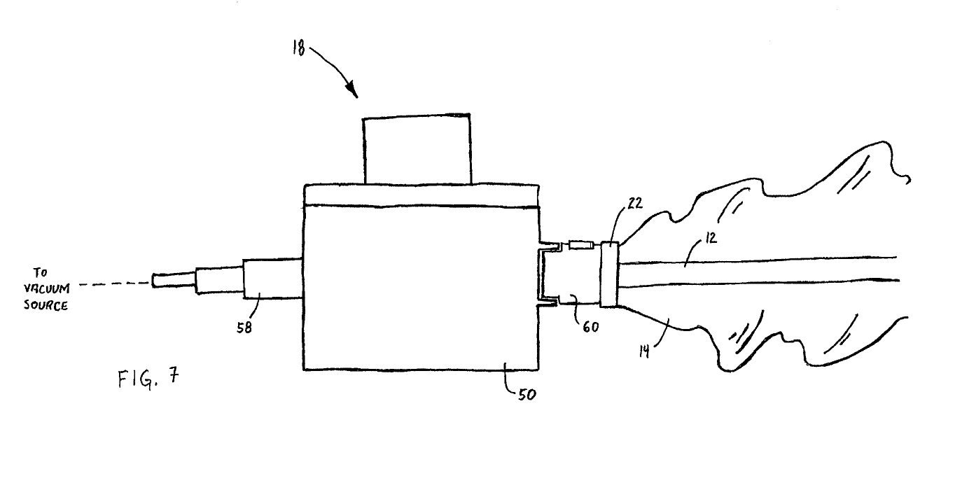

Turning now to the proximal end of closed catheter suction system 10, suction control valve 18 and its position adjacent the vacuum source is generally illustrated in FIG. 2 and is shown in enlarged detail in FIG. 7. Generally, suction control valve 18 provides a means for applying selective vacuum to catheter 12 from the vacuum source. Suction control valve 18 includes a vacuum adapter 58 which provides a connection to the vacuum source, and a lavage fitting 60 which, in part, provides a connection to suction catheter 12. Lavage fitting 60 is positioned adjacent sheath 14, where sheath 14 is secured to the exterior of lavage fitting 60 with locking ring 22. As with adapter assembly 16, locking ring 22 secures sheath 14 with an interference fit around the periphery of lavage fitting 60. Suction control valve 18 further comprises a housing 50 which accommodates additional components of the valve as more fully described below.

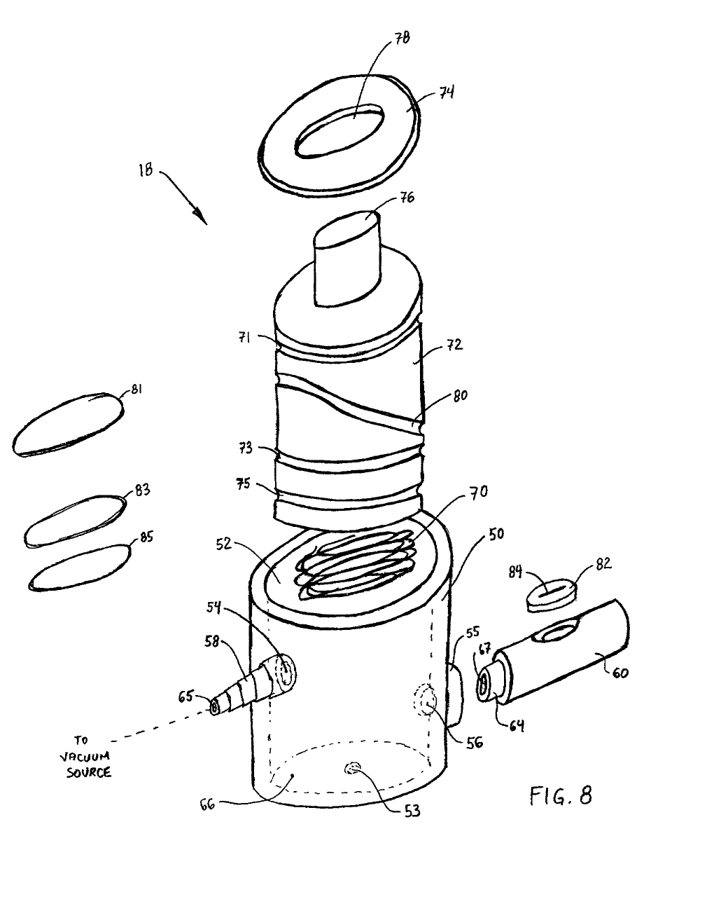

The individual components of suction control valve 18 are more clearly illustrated in FIG. 8. As shown, housing 50 defines an internal chamber 52 forming diagonally opposed vacuum and lavage apertures 54 and 56, respectively. Vacuum aperture 54 (shown in phantom) is aligned with vacuum adapter 58 which connects to the vacuum source, while lavage aperture 56 (shown in phantom) is aligned with and accommodates lavage fitting 60 which is also used to instill a lavage solution into closed catheter suction system 10 as more fully described below. Vacuum adapter 58 forms an internal channel 65 which is aligned with vacuum aperture 54 for fluid flow communication during aspiration and rinsing procedures. Similarly, lavage fitting 60 forms an internal channel 67 which is aligned with lavage aperture 54 for fluid flow communication during aspiration and rinsing procedures.

In the preferred embodiment of the present invention, vacuum adapter 58 is integral with housing 50. Alternately, vacuum adapter 58 may be a separate component and be attached to housing 50 using a variety of methods commonly known in the art.

Housing 50 further defines a female socket 55 which is integral with the construction of housing 50 in the preferred embodiment of the present invention. Female socket 55 accommodates an extension 64 formed at one end of lavage fitting 60 such that internal channel 67 is aligned with lavage aperture 54 when suction control valve 18 is fully assembled. In the preferred embodiment of the present invention, lavage fitting 60 is adhesively bonded to female socket 55, however, alternate methods such as an o-ring seated around extension 64 can also be employed to provide a fluid-tight seal around lavage aperture 56. Further, female socket 55 may be excluded all together from the construction of housing 50 such that extension 64 of lavage fitting 60 is joined to lavage aperture 56 using any number of methods commonly known in the art.

Housing 50 also defines a bleed-air hole 53 (shown in phantom) which is formed in the lower surface 66 of housing 50. Bleed-air hole 53 permits the passage of air from internal chamber 52 to the ambient air when suction control valve 18 is activated.

As further shown, internal chamber 52 houses a spring 70, a valve block 72, and a cover 74, wherein valve block 72 is positioned between cover 74 and spring 70. Valve block 72 defines a valve actuator 76 which protrudes through an aperture 78 formed in cover 74 when suction control valve 18 is fully assembled.

Valve block 72 further defines a continuous external groove 80 formed around the periphery of valve block 72. Fluids pass through external groove 80 to the vacuum source during the aspiration and rinsing procedures when suction control valve 18 is placed in the open position. When suction control valve 18 is placed in the closed position, valve block 72 prevents the passage of fluid through external groove 80 to the vacuum source. Although fluid flows through external groove 80 in the preferred embodiment of the present invention, an internal bore could alternately be formed through valve block 72 to provide for the passage of fluid to the vacuum source.

To prevent leakage of fluid, valve block 72 further defines a first o-ring groove 71, a second o-ring groove 73, and a third o-ring groove 75 in which the first, second, and third 0-rings 81, 83, and 85 are seated, respectively. First o-ring 81 prevents leakage from internal chamber 52 through cover 74 during operation of suction control valve 18. In a similar manner, second o-ring 83 and third o-ring 85 prevent leakage from internal chamber 52 through bleed-air hole 53 when suction control valve 18 is in the open and closed positions, respectively.

In the preferred embodiment of the present invention as illustrated herein, housing 50, valve block 72, and cover 74 are all cylindrical in shape, while valve actuator 76 and aperture 78 are oval in shape to prevent rotation of valve block 72 during operation of suction control valve 18. In an alternate embodiment of the present invention, housing 50, valve block 72, and cover 74 are square or rectangular in shape so as to prevent inadvertent rotation of valve block 72 during operation of suction control valve 18.

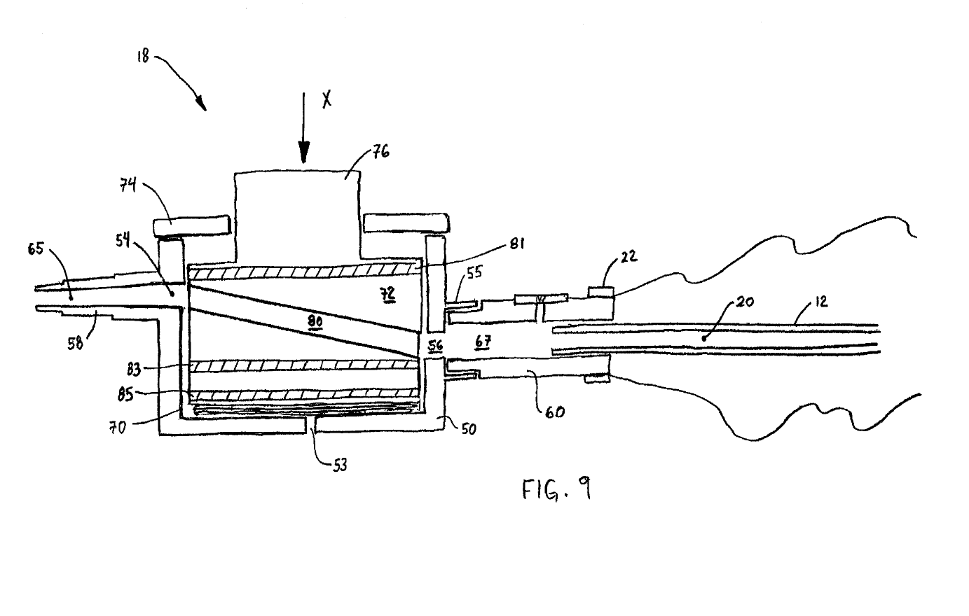

The open position of suction control valve 18 is more clearly illustrated in FIG. 9. When valve actuator 76 is fully depressed in the direction of arrow X, valve block 72 compresses spring 70, resulting in the alignment of external groove 80 with diagonally opposed apertures 54 and 56, and also with internal channels 65 and 67. Accordingly, lumen 20 of suction catheter 12 is placed in fluid flow communication with the vacuum source through external groove 80 of suction control valve 18.

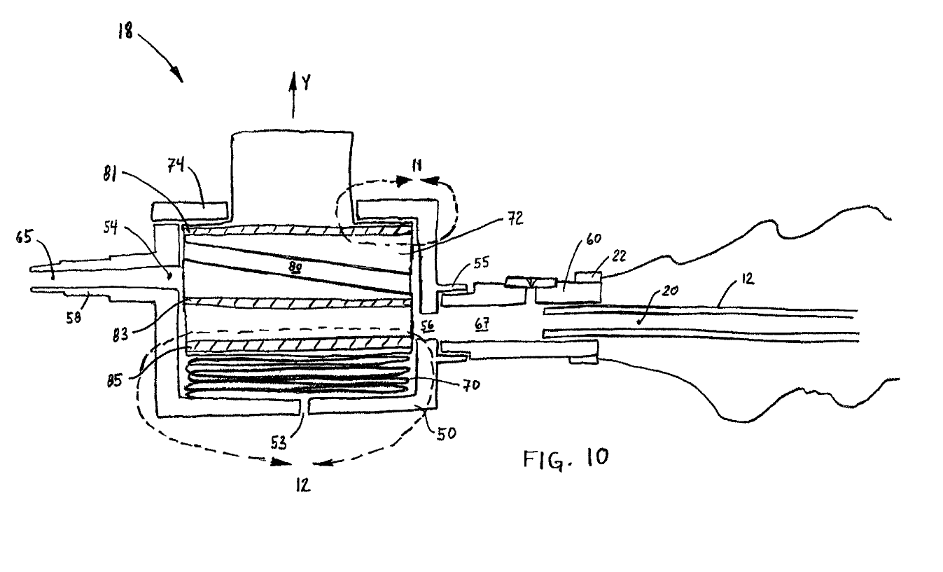

When valve actuator 76 is released, suction control valve 18 is placed in the closed position as illustrated in FIG. 10. As valve actuator 76 is released in the direction of arrow Y, spring 70 biases valve block 72 upward such that external groove 80 is brought out of alignment with diagonally opposed apertures 54 and 56. Consequently, external groove 80 is misaligned and valve block 72 occludes the flow of fluid from lumen 20 to the vacuum source, thereby placing suction control valve 18 in its closed position.

Although apertures 54 and 56 are diagonally opposed in the preferred embodiment of the present invention, the apertures may alternately be positioned directly opposing one another such that fluid flow communication through suction control valve 18 occurs along a straight line or axis from lumen 20 to the vacuum source.

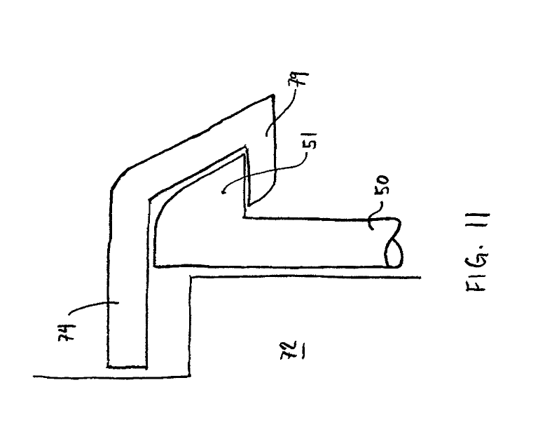

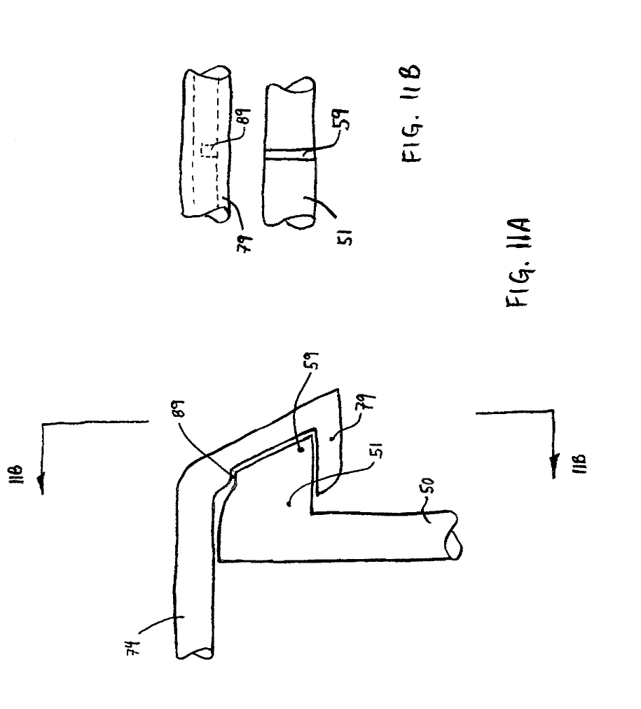

Referring to FIG. 11, cover 74 of the preferred embodiment of the present invention is a “snap-on” cover that is not easily removed once snapped into place over housing 50. As shown, cover 74 defines a flexible lip 79 which is formed around the entire periphery of cover 74. Housing 50 defines a shoulder 51 which is similarly formed around the entire periphery thereof. Consequently, lip 79 engages and locks onto shoulder 51 as cover 74 is pressed over housing 50, thereby securing valve block 72 within housing 50. Alternately, cover 74 may be adhesively bonded to housing 50 or attached by other methods commonly known in the art.

To prevent the inadvertent rotation of cover 74 after being snapped into place, additional provisions are provided within lip 79 and shoulder 51 as shown in FIGS. 11A and 11B. Cover 74 further defines a slot 89 in which a tab 59 formed on shoulder 51 is positioned. After cover 74 is snapped onto housing 50, cover 74 is rotated until tab 59 engages slot 89. When tab 59 is positioned within slot 89, cover 74 is prevented from rotating and is therefore locked into place.

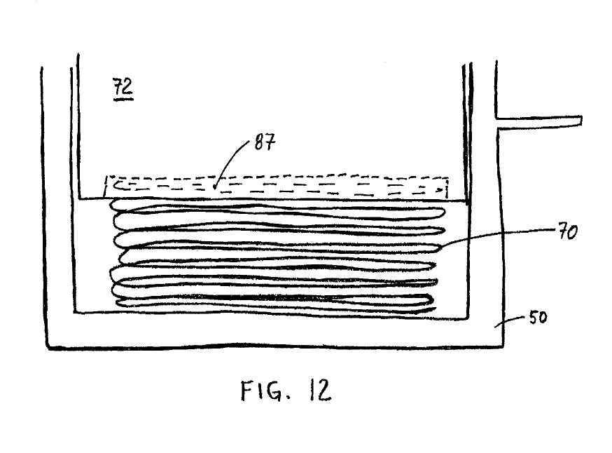

Referring to FIG. 12, valve body 72 further defines a cavity 87 which partially houses and secures spring 70. Spring 70 is fixed within cavity 87 so as to maintain its proper position and function throughout the operation of suction control valve 18.

### Lavage and Rinse Ports

As noted above, closed catheter suction system 10 includes a lavage port 17 positioned at its proximal end for introducing a lavage solution through catheter 12 and a rinse port 19 positioned at its distal end for cleansing catheter 12.

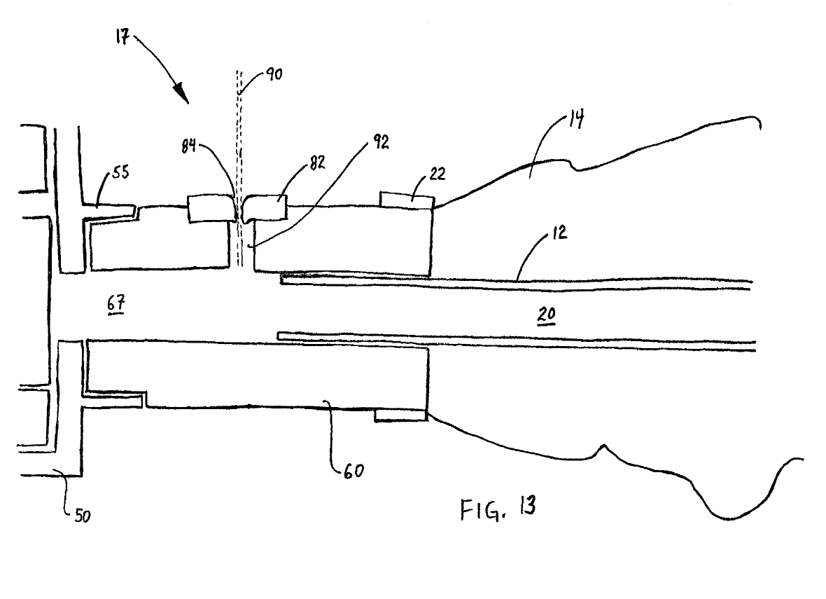

Referring to FIG. 13, lavage port 17 generally comprises lavage fitting Go and fill-valve 82. Lavage fitting 60 further defines a lavage channel 92 which is in fluid communication with lumen 20 through internal channel 67. Lavage fitting 60 also houses fill-valve 82 which defines a slit 84 to accommodate a tip 90 of a syringe (shown in phantom) or like device for instillation of a lavage solution through lumen 20. Preferably, fill-valve 82 is a sealing element disclosed in U.S. Pat. No. 5,641,184 to Mortensen and is herein incorporated by reference in its entirety.

When tip 90 is inserted through slit 84 of fill-valve 82, the flexible material of fill-valve 82 permits tip 90 to pass through lavage channel 92 and into lumen 20. Since the material of fill-valve 82 is flexible, lavage port 17 remains sealed while tip 90 is inserted therein. Accordingly, a lavage solution can pass through lavage port 17 and into lumen 20 for subsequent aspiration of the patient as more fully described herein below. The advantageous position of lavage port 17 at the proximal end of closed catheter suction system 10, as opposed to the distal end thereof, allows lavage to remain within lumen 20 rather than outside lumen 20 within sheath 14 to reduce the risk of lavage entering the ventilation circuit and instigating undesirable bacterial growth.

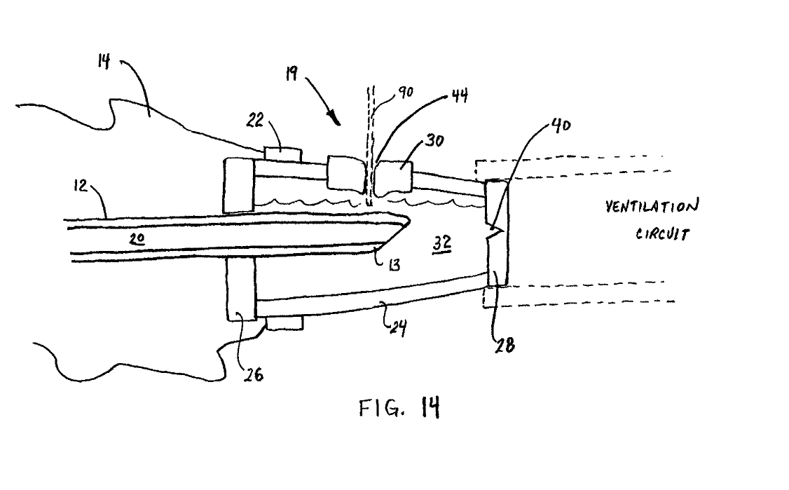

Referring now to the distal end of closed catheter suction system 10, rinse port 19 is further illustrated in FIG. 14. In the fully retracted position of closed catheter suction system 10, catheter tip 13 is disposed within rinse chamber 32, between seal 26 and one-way valve 28. The operation of rinse port 19 is similar to that of lavage port 17, in that tip 90 is inserted through fill-valve 30 and into rinse chamber 32. Because fill-valve 30 is made of a flexible material, rinse chamber housing 24 remains sealed while tip 90 is positioned within rinse chamber 32. Rinse solution is then instilled through tip 90 and into rinse chamber 32, where the rinse solution collects and is in fluid communication with lumen 20. Rinse solution can then be evacuated through tip 13 and through lumen 20 when suction control valve 18 is activated as previously described in association with FIG. 9. Accordingly, when valve actuator 76 is depressed, the rinse solution flows through lumen 20 and suction control valve 18, and into the vacuum source for proper disposal. Although fluid may not be in direct contact with tip 13, the vacuum created through lumen 20 expunges all remaining fluid remaining within rinse chamber 32.

As a result, the rinsing of lumen 20 is isolated from the ventilation circuit which allows full ventilator volume to be delivered to the patient without interruption and reduces the increased risk of hypoxia during the rinse procedure. Additionally, an isolated rinse procedure prevents aspirated secretions from re-entering patient passageways, reduces the risk of accidental patient suctioning, and prevents accidental leakage of volume from the ventilation circuit.

### System Operation

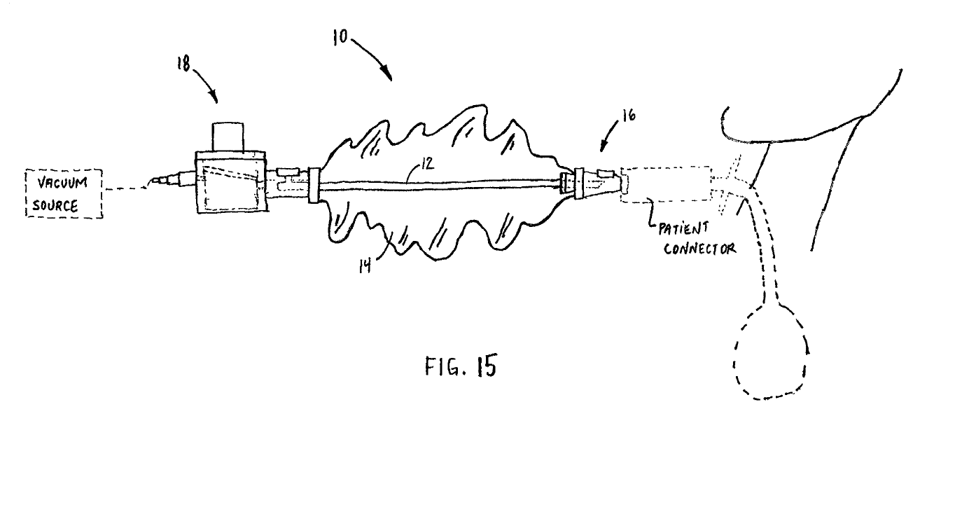

The aspiration procedure begins with the user positioning closed catheter suction system 10 as illustrated in FIG. 15. The user holds suction control valve 18 at the proximal end with one hand and stabilizes the patient connector at the distal end with the other hand. As shown, adapter assembly 16 is operatively engaged with the patient connector, suction control valve 18 is operatively connected to the vacuum source, and closed catheter suction system 10 is in its fully retracted position.

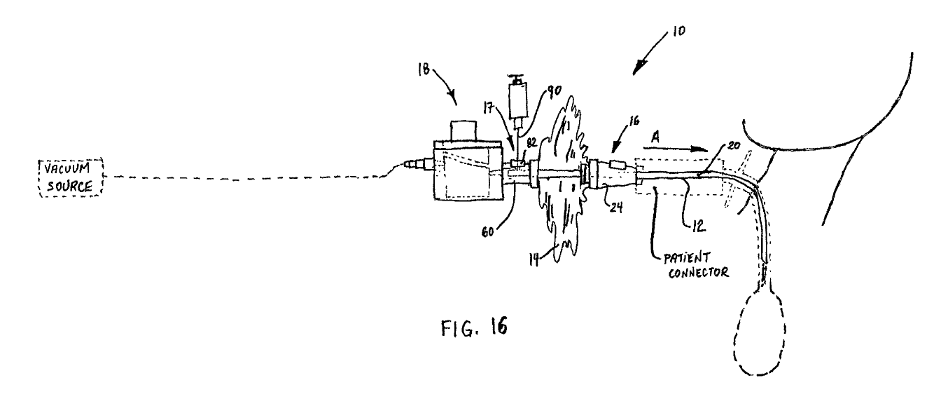

Referring to FIG. 16, the user then advances the proximal end of closed catheter suction system 10 in the direction of arrow A, towards the patient. Consequently, catheter 12 passes through rinse chamber housing 24 and the patient connector, and into the trachea of the patient where secretions subsist. As shown, sheath 14 is appressed between suction control valve 18 and adapter assembly 16 while maintaining a sealed connection between the same.

The user then inserts tip 90 of a syringe containing a lavage solution into fill-valve 82 of lavage port 17 such that the lavage solution can be instilled through lumen 20. Accordingly, lavage flows through lumen 20 in the direction of arrow A until a sufficient quantity of lavage loosens the secretions in the passageway of the patient.

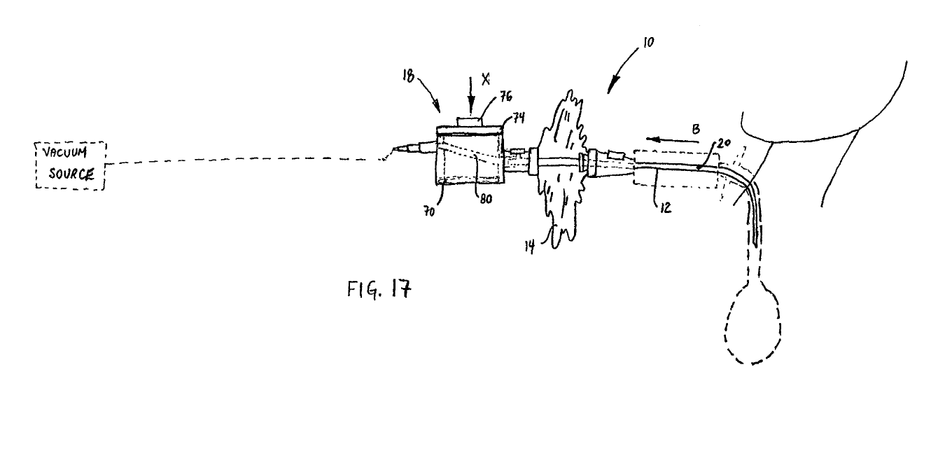

Referring to FIG. 17, the lavage and secretions are then aspirated from the patient with the activation of suction control valve 18. Once the vacuum source is activated, the user depresses valve actuator 76 in the direction of arrow X, and as a result, spring 70 becomes compressed and external groove 80 is aligned such that lumen 20 is in fluid communication with the vacuum source. Accordingly, the suction applied by the vacuum source aspirates the lavage and secretions from the trachea of the patient in the direction of arrow B through lumen 20 and external groove 80, and into the vacuum source for proper disposal.

Advantageously, since lavage port 17 is positioned at the proximal end of closed catheter suction system 10 as opposed to the distal end thereof, the risk of lavage entering the ventilation circuit is substantially reduced. As a result, the risk of undesirable moisture accumulation within the ventilation circuit which could lead to bacterial growth is greatly reduced or eliminated.

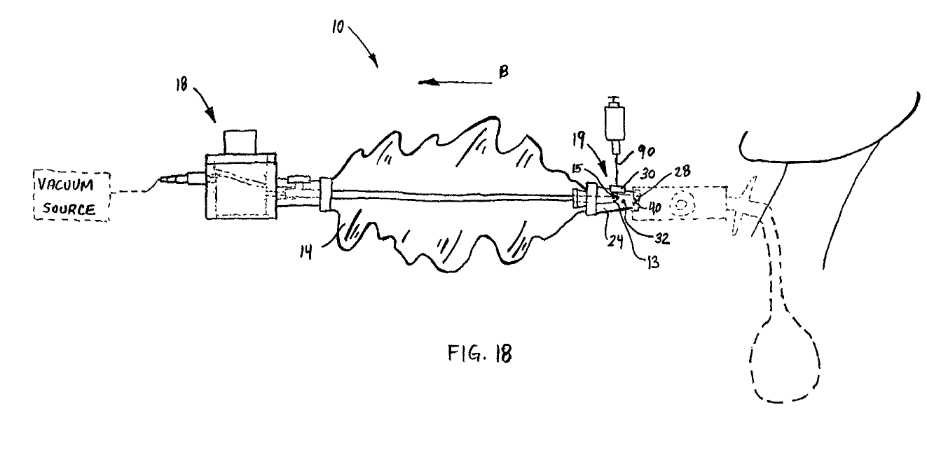

After the patient is properly aspirated, the proximal end of closed catheter suction system 10 is drawn back as illustrated in FIG. 18, in the direction of arrow B, for the subsequent rinse procedure. To rinse closed catheter suction system 10, catheter 12 is first drawn back through one-way valve 28 to its fully retracted position such that catheter tip 13 and openings 15 are disposed within rinse chamber 32. Accordingly, tapered aperture 40 closes and the ventilation circuit is sealed once again to allow full ventilator volume to be delivered to the patient. To cleanse lumen 20 of residual secretions, the user inserts tip 90 of a syringe containing a rinse solution through fill-valve 30 of rinse port 19 and the rinse solution is instilled into and collects within rinse chamber 32.

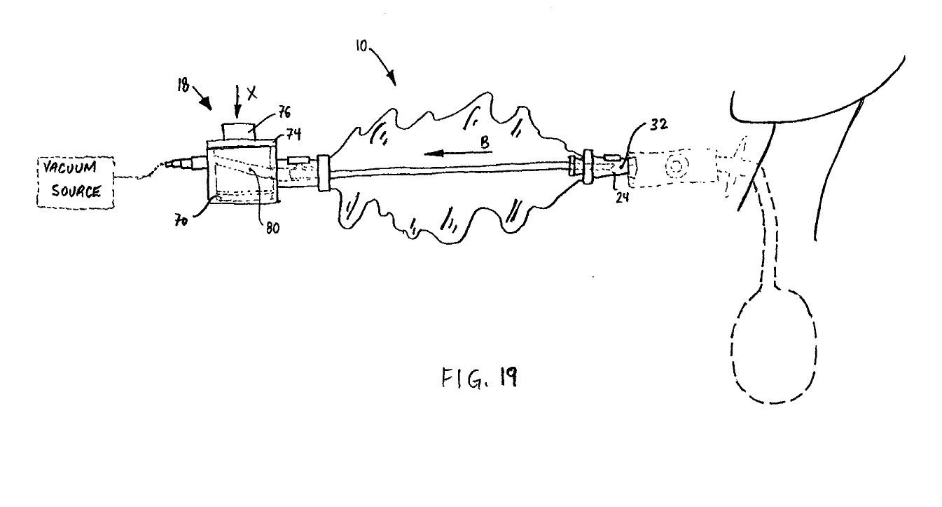

After a sufficient amount of rinse solution has filled rinse chamber 32, the rinse solution is suctioned back through lumen 20 to clear any residual secretions by activating suction control valve 18 according to FIG. 19. As shown, valve actuator 76 is depressed in the direction of arrow X, and as a result, spring 70 becomes compressed and external groove 80 is properly aligned such that lumen 20 is placed in fluid communication with the vacuum source. Once so aligned, the vacuum source suctions the rinse solution and any residual secretions in the direction of arrow B through lumen 20 and external groove 80, and into the vacuum source for proper disposal.

Since the rinse procedure is isolated from the ventilation circuit, loss of ventilator volume is prevented, infectious secretions are prevented from re-entering passageways of the patient, accidental patient suctioning is obviated, and the mishandling of closed catheter suction system 10 cannot result in accidental leakage from the ventilation system.

Many of the components of the present invention noted above are fabricated from a lightweight material such as plastic to facilitate ease of use and handling. Preferably, catheter 12 is made from a clear flexible material such as polyvinyl chloride (PVC) while sheath 14 is also made from a clear flexible material, of much thinner gauge, such as a low density polypropylene. Sheath locking rings 22 are preferably made of polypropylene.

The present invention further contemplates that rinse chamber housing 24 of adapter assembly 16 is preferably made of a clear polypropylene. Seal 26, one-way valve 28, and fill-valve 30 are preferably a silicon rubber material.

The components of suction control valve 18, such as housing 50, valve block 72, cover 74, vacuum adapter 58, and lavage fitting 60 are preferably made of a plastic material such as Nylon-66. Preferably, fill-valve 82 and gaskets 66 and 68 are made of a rubber-vinyl material.

In view of the foregoing description of the present invention and practical embodiments it will be seen that the several objects of the invention are achieved and other advantages are attained. The embodiments and examples were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting.

The breadth and scope of the present invention should not be limited by any of the above-described exemplary embodiments, but should be defined only in accordance with claims of the application and their equivalents.

What is claimed is:

1. A catheter suction system comprising:
a catheter having a proximal end and a distal end, said catheter defining a lumen therethrough which communicates with a tip formed at said distal end of said catheter;
an adapter assembly slidably engageable with said distal end of said catheter, said adapter assembly comprising a rinse chamber isolated from a ventilation circuit; and
a suction control valve secured to said proximal end of said catheter and in communication with a vacuum source;
wherein when said tip is disposed within said rinse chamber a rinse solution is then instilled into said rinse chamber; and
when said suction control valve is activated, the rinse solution is aspirated through said tip and said lumen and into the vacuum source to cleanse said catheter without interrupting the ventilation circuit.

2. The catheter suction system according to claim 1 further comprising:
a sheath encasing said catheter;
wherein said sheath is secured at one end to said suction control valve and secured at the other end to said adapter assembly.

3. The catheter suction system according to claim 2 wherein said rinse chamber defines a rinse housing, said rinse housing having a proximal end and a distal end, and said adapter assembly further comprises:
a seal disposed at said proximal end of said rinse housing to seal said rinse chamber from said sheath; and
a one-way valve disposed at said distal end of said rinse housing to seal said rinse chamber from the ventilation circuit.

4. The catheter suction system according to claim 3 wherein said one-way valve further comprises:
a tapered aperture which abuts said catheter and seals said rinse chamber from the ventilation circuit.

5. The catheter suction system according to claim 4 wherein said adapter assembly further comprises:
a fill-valve in fluid communication with said rinse chamber, said fill-valve further comprising a slit to accommodate an injection means for instillation of a rinse solution into said rinse chamber.

6. The catheter suction system according to claim 2 further comprising:
a first locking collar which secures said one end of said sheath to said suction control valve; and
a second locking collar which secures said other end of said sheath to said adapter assembly.

7. The catheter suction system according to claim 1 further comprising:
a lavage port attached to said suction control valve and adjacent said proximal end of said catheter, said lavage port being in fluid communication with said lumen;
wherein when said catheter is advanced into a passageway of a patient and a lavage solution is instilled through said lavage port such that the lavage solution flows through said lumen to said tip and into the passageway of the patient; and
wherein when said suction control valve is activated, secretions and the lavage solution are aspirated from the passageway of the patient through said lumen and into the vacuum source.

8. The catheter suction system according to claim 7 wherein said lavage port further comprises:
a lavage fitting attached to said suction control valve, said lavage fitting comprising a lavage channel in direct fluid communication with an internal channel, said internal channel being in direct fluid communication with said lumen.

9. The catheter suction system according to claim 8 wherein said lavage fitting further comprises:
a fill-valve in direct fluid communication with said lavage channel, said fill-valve comprising a slit to accommodate an injection means for instillation of a lavage solution into said lumen.

10. The catheter suction system according to claim 1 wherein said suction control valve further comprises:
a housing, said housing defining an internal chamber and opposed vacuum and lavage apertures;
a vacuum adapter attached to said housing, said vacuum adapter forming an internal channel aligned with said vacuum aperture;
a spring disposed within said housing;
a valve block disposed adjacent said spring within said housing, said valve block defining an external groove and upper and lower ends; and
a cover disposed adjacent said valve block;
wherein when said valve block is fully depressed against said spring, said external groove is properly aligned with said vacuum aperture and said lavage aperture, thereby permitting the aspiration of secretions, lavage solution, and rinse solution from said lumen into the vacuum source.

11. The catheter suction system according to claim 10 wherein said upper end of said valve block forms an actuator, and said cover further comprises an aperture, wherein said actuator protrudes through said aperture for engagement by a user.

12. The catheter suction system according to claim 11 wherein said actuator and said aperture are oval shaped to prevent rotation of said valve block during operation of said suction control valve.

13. The catheter suction system according to claim 11 wherein said housing, said valve block, and said cover are rectangular shaped to prevent rotation of said valve block during operation of said suction control valve.

14. The catheter suction system according to claim 10 wherein said valve block further defines a cavity formed in said lower end of said valve block wherein said spring is seated.

15. The catheter suction system according to claim 10 wherein said housing further defines a lower surface, said lower surface defining a bleed-air hole to permit the passage of air from said internal chamber to the ambient air during operation of said suction control valve.

16. The catheter suction system according to claim 15 wherein said suction control valve further comprises:
a first o-ring;
a second o-ring; and
a third o-ring, and wherein said valve block further comprises:
a first o-ring groove;
a second o-ring groove; and
a third o-ring groove,
wherein said first o-ring is disposed within said first o-ring groove to prevent leakage from said housing through said cover;
said second o-ring is disposed within said second o-ring groove to prevent leakage from said housing through said bleed-air hole when said valve block is fully depressed; and
said third o-ring is disposed within said third o-ring groove to prevent leakage from said housing through said bleed-air hole when said valve block is not fully depressed.

17. The catheter suction system according to claim 10 wherein said cover further comprises a lip and said housing further comprises a shoulder, wherein said lip engages said shoulder to secure said cover to said housing.

18. The catheter suction system according to claim 17 wherein said lip further comprises a slot and said shoulder further comprises a tab, wherein said tab engages said shoulder to prevent said cover from rotating.

19. The catheter suction system according to claim 10 further comprising:
a lavage port secured to said suction control valve and adjacent said proximal end of said catheter, said lavage port being in fluid communication with said lumen;
wherein when said catheter is advanced into a passageway of a patient and a lavage solution is instilled through said lavage port, the lavage solution flows through said lumen to said tip and into the passageway of the patient; and
wherein when said suction control valve is activated, secretions and the lavage solution are aspirated from the passageway of the patient through said lumen and into the vacuum source.

20. The catheter suction system according to claim 19 wherein said lavage port further comprises:
a lavage fitting attached to said suction control valve, said lavage fitting further comprising a lavage channel in direct fluid communication with an internal channel, said internal channel being in direct fluid communication with said lumen and said lavage aperture.

21. The catheter suction system according to claim 19 wherein said lavage fitting further comprises:
a fill-valve in direct fluid communication with said lavage channel, said fill-valve further comprising a slit to accommodate an injection means for instillation of a lavage solution into said lumen.

22. The catheter suction system according to claim 20 wherein:
said housing further comprises a female socket and said lavage fitting further comprises an extension, wherein said extension is attached within said female socket to secure said lavage fitting to said housing.

23. The catheter suction system according to claim 1 wherein said suction control valve further comprises:
a housing, said housing defining an internal chamber and opposed vacuum and lavage apertures;
a vacuum adapter attached to said housing, said vacuum adapter forming an internal channel aligned with said vacuum aperture;
a spring disposed within said housing;
a valve block disposed adjacent said spring within said housing, said valve block defining an internal bore; and
a cover disposed adjacent said valve block;
wherein when said valve block is fully depressed against said spring, said internal bore is properly aligned with said vacuum aperture and said lavage aperture, thereby permitting the aspiration of secretions, lavage solution, and rinse solution from said lumen into the vacuum source.

24. A method for rinsing a catheter suction system without interrupting a ventilation circuit, said method comprising the steps of:
(a) providing a catheter suction system comprising:
a catheter having a proximal end and a distal end, said catheter defining a lumen therethrough which communicates with a tip formed at said distal end of said catheter;
an adapter assembly slidably engageable with said distal end of said catheter, said adapter assembly comprising a rinse chamber isolated from a ventilation circuit; and
a suction control valve secured to said proximal end of said catheter and in communication with a vacuum source;
(b) retracting said catheter suction system towards said proximal end until said tip is positioned within said rinse chamber;
(c) instilling a rinse solution into said rinse chamber, wherein said rinse solution flows into said lumen and through said tip; and
(d) activating said suction control valve, wherein said rinse solution is aspirated through said lumen and into said vacuum source without interrupting the ventilation circuit.

25. A method for aspirating secretions from a passageway of a patient with a catheter suction system and rinsing said catheter suction system without interrupting a ventilation circuit, said method comprising the steps of:
(a) providing a catheter suction system comprising:
a catheter having a proximal end and a distal end, said catheter defining a lumen therethrough which communicates with a tip formed at said distal end of said catheter;
an adapter assembly slidably engageable with said distal end of said catheter, said adapter assembly comprising a rinse port isolated from a ventilation circuit;
a suction control valve secured to said proximal end of said catheter and in communication with a vacuum source; and
a lavage port secured to said suction control valve and adjacent said proximal end of said catheter, said lavage port being in fluid communication with said lumen.
(b) advancing said catheter suction system towards said distal end, wherein said catheter slides through said adapter assembly until said tip is positioned within a passageway of a patient having secretions;
(c) instilling a lavage solution through said lavage port, wherein said lavage solution flows through said lumen to said tip within the passageway;
(d) activating said suction control valve, wherein said lavage solution and said secretions are aspirated from the patient;
(e) retracting said closed catheter suction system towards said proximal end until said tip is positioned within said rinse chamber;
(f) instilling a rinse solution into said rinse chamber, wherein said rinse solution flows into said lumen through said tip;
(g) activating said suction control valve, wherein said rinse solution is aspirated through said lumen and into said vacuum source without interrupting the ventilation circuit.
